(19) United States
(12) Patent Application Publication
(10) Pub. No.: US 2025/0058140 A1
(43) Pub. Date: Feb. 20, 2025

(54) PROGRAMMABLE GRAPHIC PROJECTION LIGHT SOURCE SYSTEM FOR OPHTHALMOLOGY

(71) Applicant: EYE AND EAR NOSE AND THROAT HOSPITAL AFFILIATED TO FUDAN UNIVERSITY, Shanghai (CN)

(72) Inventors: Jinhai Huang, Shanghai (CN); Xingtao Zhou, Shanghai (CN); Jing Zhao, Shanghai (CN); Cuizhi Wang, Shanghai (CN); Yang Shen, Shanghai (CN); Zhi Chen, Shanghai (CN)

(21) Appl. No.: 18/711,617

(22) PCT Filed: Apr. 15, 2022
(86) PCT No.: PCT/CN2022/087000

§ 371 Date: May 20, 2024

(30) Foreign Application Priority Data
Dec. 10, 2021 (CN) 202111504976.9

(51) Int. Cl. A61N 5/06

(52) U.S. Cl. CPC A61N 5/0613; A61N 2005/0632; A61N 2005/0643; A61N 2005/0661; A61N 2005/0665

(57) ABSTRACT

A programmable graphic projection light source system for ophthalmology includes a light source, a flexible screen, a reflective coating, and a light-shielding plate that is located between the light source and a pupil. The light source irradiates light away from the light-shielding plate. The flexible screen is located opposite the light-shieling plate crossing the light source. The reflective coating is located opposite the light source crossing the flexible screen. After the light is reflected by a central, spherical face portion of the reflective coating, a part of the light transferring a pattern from the flexible screen is blocked by the light-shielding plate, and another part of the light irradiates orthographically onto a periphery of the pupil. After the light is reflected by a plane portion of the reflective coating, a part of the light transferring the pattern irradiates obliquely onto the pupil.

### BACKGROUND OF THE INVENTION

### 1. Technical Field

The present invention relates to medical instruments, and more particularly to a programmable graphic projection light source system for ophthalmology.

### 2. Description of Related Art

Referring to FIG. 1, during ophthalmic treatment, it is a common practice that ultraviolet is casted to a cornea of a patient with a specific pattern (that can be personalized by adjusting energy magnitude). Specifically, the pattern is projected to the corneal region above the pupil and the corneal region above the periphery of the pupil (the iris) for providing treatment for local corneal diseases. Traditionally, the projection is directly made on to the pupil using a projection device. However, this means that ultraviolet irradiated by the projection device irradiate the fundus orthographically, which can cause injury to the fundus over time.

### SUMMARY OF THE INVENTION

In view of the shortcomings of the prior art, the objective of the present invention is to provide a programmable graphic projection light source system for ophthalmology, which prevents ultraviolet from irradiating the fundus of a patient orthographically, so as to protect the fundus during corneal treatment.

To achieve the foregoing objective, the present invention proposes technical schemes as described below. A programmable graphic projection light source system for ophthalmology comprises a housing in which a light source, a flexible screen, a reflective coating, and a light shielding plate are arranged, the light shielding plate being located between the light source and a pupil, the light source irradiating light in a direction away from the light shielding plate, the flexible screen being located opposite the light shielding plate crossing the light source, the reflective coating being located opposite the light source crossing the flexible screen, the reflective coating comprising a spherical face portion located centrally and a plane portion surrounding the spherical face portion, whereby after the light irradiated by the light source is reflected by the spherical face portion, a part of the light transferring a pattern shown in the flexible screen is blocked by the light shielding plate, and another part of the light irradiates orthographically onto a periphery of the pupil, and after the light irradiated by the light source is reflected by the plane portion, a part of the light transferring the pattern shown in the flexible screen irradiates obliquely onto the pupil.

With the technical scheme described previously, the path of the light is altered as compared to the irradiation direction of the light source such that the light is first reflected by the reflective coating and then projects the pattern to the eye. In this way, the light otherwise would irradiate the pupil directly is blocked by the light shielding plate, so the patient is protected as no ultraviolet can reach the fundus. Meanwhile, as the rest of the light, a part is redirected to irradiate the periphery of the pupil directly and a part irradiates the pupil obliquely, thereby ensuring completeness of the projected pattern, which is the guarantee of accurate diagnosis or treatment.

The present invention is further configured that: the light shielding plate is provided with a diameter adjusting device, which comprises adjusting slide blocks, adjusting press blocks, and a first micro servo motor, the light shielding plate having a cross slide located on a radial surface thereof, the cross slide comprising an intersection portion located centrally and four branch portions distributed along a circumference of the intersection portion, the adjusting slide blocks being in one-to-one correspondence with the branch portions and slidable along the corresponding branch portions, the light shielding plate being axially provided with an adjusting slide that is communicated with the intersection portion and is for the adjusting press blocks to slide therein, the first micro servo motor being fixed to the light shielding plate and serving to drive a first adjusting screw that is in threaded fit with the adjusting press blocks, so that when the first adjusting screw rotates, the adjusting press blocks move along the adjusting slide, the adjusting press blocks having four lateral sides thereof formed with pressing ramps each pressing on one of the adjusting slide blocks, the adjusting slide blocks each being provided with a propping ramp that fits the corresponding pressing ramp and moves along the corresponding branch portion when being pressed, a deformable blocking portion being provided between the lateral sides of the adjacent two adjusting press blocks, the light shielding plate having an outer side provided with a receiving recess for receiving the deformable blocking portions that retract into the light shielding plate, each of the branch portions having a lateral surface provided with a radially extending returning groove, each of the adjusting slide blocks being provided with a returning bulge that moves along the returning groove, and a compression spring being arranged between the returning bulge and the returning groove for returning the adjusting slide block toward the adjusting press block.

As the size of the pupil is different from person to person, it is desired that the light shielding plate has a matching diameter. With the technical scheme described previously, the diameter of the light shielding plate defines the minimum dimension of the light shielding plate, and a diameter adjusting device may be used to increase the diameter. The diameter adjusting device when working with the first micro servo motor provides the possibility to precisely enlarge the blocking area according to practical needs, further protecting the patient form ultraviolet.

The present invention is further configured that it comprises a reflection angle adjusting device, which comprises a second micro servo motor, a screw seat, a screw nut, and adjusting arms, the second micro servo motor being fixed to the housing and abutting against a side of the reflective coating opposite the flexible screen, the second micro servo motor driving a second adjusting screw to move in a direction of light irradiating directly onto the pupil, the screw seat being fixed to the housing and in rotational fit with an end of the second adjusting screw opposite the second micro servo motor, the screw nut being mounted around the second adjusting screw and moving along an axis of the second adjusting screw when the second adjusting screw rotates, the screw nut being peripherally provided with at least four circumferentially distributed links, the adjusting arms being in one-to-one correspondence with the links, and a center of the adjusting arm being pivotally connected to an end portion of the corresponding link, the link having one end pivotally connected to the screw seat and an opposite end pivotally connected to the side of the reflective coating opposite the flexible screen.

It is known that the shape of the pupil is different from person to person. With the technical scheme described previously, the range covered by the light directly irradiating the pupil from the spherical face portion and the angle of the light obliquely irradiating the pupil from the plane portion can be adapted to the patient by changing the shape of the spherical face portion using the reflection angle adjusting device. In addition, with the precise driving provided by the second micro servo motor, the accuracy of acquired data can be further improved.

The present invention is further configured that the flexible screen is connected to an image input device in a wired or wireless manner.

With the technical scheme described previously, the image input device may simulate different patterns through programming, thereby projecting different patterns to the eye. This eliminates the defects of the known devices about incapability of changing patterns, and enables the disclosed system to simulate different scenes, thereby obtaining more comprehensive data.

### BRIEF DESCRIPTION OF THE DRAWINGS

FIG. 1 shows a traditional arrangement;

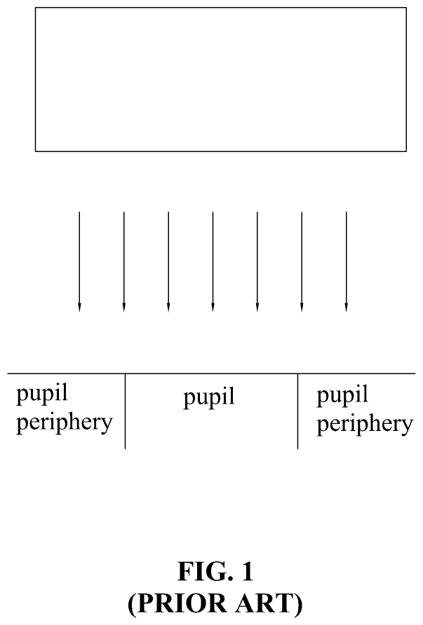

FIG. 2 is a schematic drawing of a system of the present invention;

FIG. 3 is a cross-sectional view of a light shielding plate of the system; and

FIG. 4 is a cross-sectional view taken along line A-A of FIG. 3.

### DETAILED DESCRIPTION OF THE INVENTION

To make the foregoing objectives, features, and advantages of the present invention clearer and more understandable, the following description will be directed to some embodiments as depicted in the accompanying drawings to detail the technical schemes disclosed in these embodiments. It is, however, to be understood that the embodiments referred herein are only a part of all possible embodiments and thus not exhaustive. Based on the embodiments of the present invention, all the other embodiments can be conceived without creative labor by people of ordinary skill in the art, and all these and other embodiments shall be encompassed in the scope of the present invention.

It is to be noted that throughout the description of the present invention, orientations or relative positions described using terms such as “center,” “up,” “down,” “left,” “right,” “vertical,” “horizontal,” “inner,” and “outer” are based on the orientations or relative positions shown in the accompany drawings, and are merely for concise writing, but not intended to represent or imply that the described devices or elements must have or be operated in such orientations or relative positions, thus by no means being limiting to the present invention. In addition, terms like “first,” “second,” and “third” are merely descriptive, and shall not be construed as representing or implying relative importance among the described devices or elements.

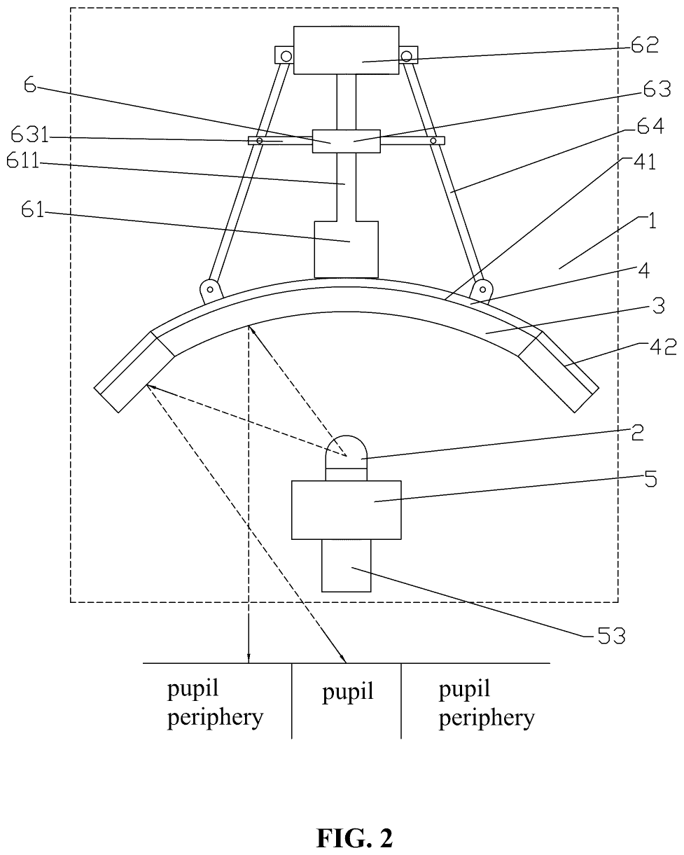

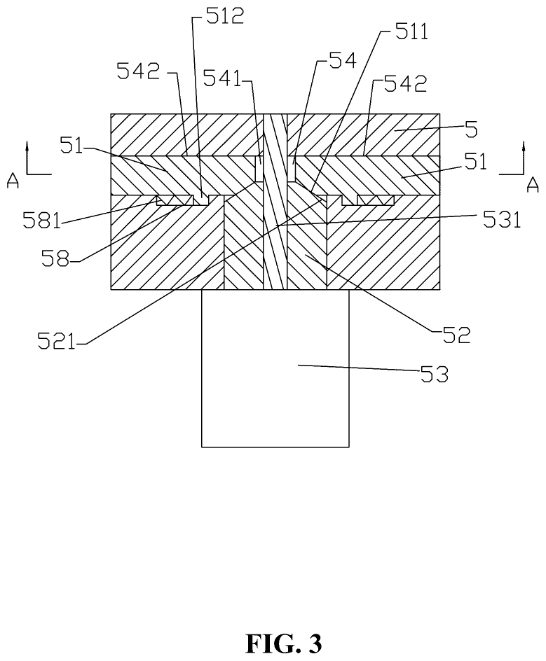

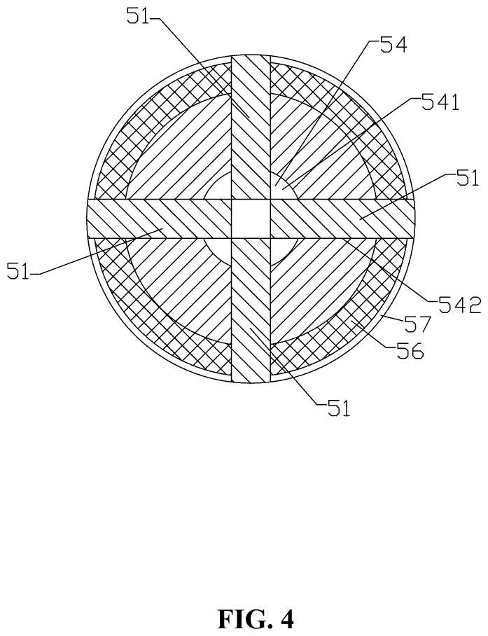

As shown in FIG. 2 through FIG. 4, the present invention discloses a programmable graphic projection light source system for ophthalmology. The programmable light source system comprises a housing 1 in which a light source 2, a flexible screen 3, a reflective coating 4, and a light shielding plate 5 are arranged. The light source 2 may typically be an LED. The light shielding plate 5 is located between the light source 2 and a pupil of a patient. The light source 2 irradiates light away from the light shielding plate 5. The flexible screen 3 is located opposite the light shielding plate 5 crossing the light source 2. The reflective coating 4 is located opposite the light source 2 crossing the flexible screen 3. The reflective coating 4 comprises a spherical face portion 41 located centrally and a plane portion 42 surrounding the spherical face portion 41. After the light irradiated by the light source 2 is reflected by the spherical face portion 41, a part of the light transferring a pattern shown in the flexible screen 3 is blocked by the light shielding plate 5 while another part of the light irradiates orthographically onto the periphery of the pupil. After the light irradiated by the light source 2 is reflected by the plane portion 42, a part of the light transferring the pattern shown in the flexible screen 3 irradiates obliquely onto the pupil. Thereby, the path of the light is altered as compared to the irradiation direction of the light source 2 such that the light is first reflected by the reflective coating 4 and then projects the pattern to the eye. In this way, the light otherwise would irradiate the pupil directly is blocked by the light shielding plate 5, so the patient is protected as no ultraviolet can reach the fundus. Meanwhile, as the rest of the light, a part is redirected to irradiate the periphery of the pupil directly and a part irradiates the pupil obliquely, thereby ensuring completeness of the projected pattern, which is the guarantee of accurate diagnosis or treatment.

The light shielding plate 5 is provided with a diameter adjusting device, which comprises adjusting slide blocks 51, adjusting press blocks 52, and a first micro servo motor 53. The light shielding plate 5 has a cross slide 54 located on a radial surface. The cross slide 54 comprises an intersection portion 541 located centrally and four branch portions 542 distributed along a circumference of the intersection portion 541. The adjusting slide blocks 51 are in one-to-one correspondence with the branch portions 542 and slidable along the corresponding branch portions 542. The light shielding plate 5 is axially provided with an adjusting slide 55 that is communicated with the intersection portion 541 and is for the adjusting press blocks 52 to slide therein. The first micro servo motor 53 is fixed to the light shielding plate 5 and serves to drive a first adjusting screw 531 that is in threaded fit with the adjusting press blocks 52, so that when the first adjusting screw 531 rotates, the adjusting press blocks 52 move along the adjusting slide 55. The adjusting press blocks 52 each have four lateral sides thereof formed with pressing ramps 521 each pressing on one of the adjusting slide blocks 51. The adjusting slide blocks 51 are each provided with a propping ramp 511 that fits the corresponding pressing ramp 521 and moves along the corresponding branch portion 542 when being pressed. A deformable blocking portion 56 is provided between the lateral sides of the adjacent two adjusting press blocks 52. The deformable blocking portions 56 are made of an elastic material and serves to fill gaps appearing when the adjusting press blocks 52 shift, so as to complete the round blocking area. The light shielding plate 5 has an outer side provided with a receiving recess 57 for receiving the deformable blocking portions 56 that retract into the light shielding plate 5. Each of the branch portions 542 has a lateral surface provided with a radially extending returning groove 58. Each of the adjusting slide blocks 51 is provided with a returning bulge 512 that moves along the returning groove 58. A compression spring 581 is arranged between the returning bulge 512 and the returning groove 58 for returning the adjusting slide block 51 toward the adjusting press block 52. As the size of the pupil is different from person to person, it is desired that the light shielding plate 5 has a matching diameter. The diameter of the light shielding plate 5 defines the minimum dimension of the light shielding plate 5, and a diameter adjusting device may be used to increase the diameter. The diameter adjusting device when working with the first micro servo motor 53 provides the possibility to precisely enlarge the blocking area according to practical needs, further protecting the patient form ultraviolet.

The disclosed system further comprises a reflection angle adjusting device 6, which comprises a second micro servo motor 61, a screw seat 62, a screw nut 63, adjusting arms 64. The second micro servo motor 61 is fixed to the housing 1 and abuts against a side of the reflective coating 4 opposite the flexible screen 3. The second micro servo motor 61 drives a second adjusting screw 611 to move in a direction of light irradiating directly onto the pupil. The screw seat 62 is fixed to the housing 1 and in rotational fit with an end of the second adjusting screw 611 opposite the second micro servo motor 61. The screw nut 63 is mounted around the second adjusting screw 611 and moves along an axis of the second adjusting screw 611 when the second adjusting screw 611 rotates. The screw nut 63 is peripherally provided with at least four circumferentially distributed links 631. The adjusting arms 64 are in one-to-one correspondence with the links 63. The center of the adjusting arm 64 is pivotally connected to an end portion of the corresponding link 63. The link has one end pivotally connected to the screw seat 62 and an opposite end pivotally connected to the side of the reflective coating 4 opposite the flexible screen 3. As the shape of the pupil is different from person to person, it is desired that the range covered by the light directly irradiating the pupil from the spherical face portion 41 and the angle of the light obliquely irradiating the pupil from the plane portion 42 can be adapted to the patient by changing the shape of the spherical face portion 41 using the reflection angle adjusting device 6. In addition, with the precise driving provided by the second micro servo motor 61, the accuracy of acquired data can be further improved.

The flexible screen 3 is connected to an image input device in a wired or wireless manner. The image input device may simulate different patterns through programming, thereby projecting different patterns to the eye. This eliminates the defects of the known devices about incapability of changing patterns, and enables the disclosed system to simulate different scenes, thereby obtaining more comprehensive data.

Additionally, the disclosed system has a controller that enables collaboration among the foregoing components.

What is claimed is:

1. A programmable graphic projection light source system for ophthalmology, the system comprising a housing in which a light source, a flexible screen, a reflective coating, and a light shielding plate are arranged, the light shielding plate being located between the light source and a pupil, the light source irradiating light in a direction away from the light shielding plate, the flexible screen being located opposite the light shielding plate crossing the light source, the reflective coating being located opposite the light source crossing the flexible screen, the reflective coating comprising a spherical face portion located centrally and a plane portion surrounding the spherical face portion, whereby after the light irradiated by the light source is reflected by the spherical face portion, a part of the light transferring a pattern shown in the flexible screen is blocked by the light shielding plate, and another part of the light irradiates orthographically onto a periphery of the pupil, and after the light irradiated by the light source is reflected by the plane portion, a part of the light transferring the pattern shown in the flexible screen irradiates obliquely onto the pupil.

2. The programmable graphic projection light source system of claim 1, wherein the light shielding plate is provided with a diameter adjusting device, which comprises adjusting slide blocks, adjusting press blocks, and a first micro servo motor, the light shielding plate having a cross slide located on a radial surface thereof, the cross slide comprising an intersection portion located centrally and four branch portions distributed along a circumference of the intersection portion, the adjusting slide blocks being in one-to-one correspondence with the branch portions and slidable along the corresponding branch portions, the light shielding plate being axially provided with an adjusting slide that is communicated with the intersection portion and is for the adjusting press blocks to slide therein, the first micro servo motor being fixed to the light shielding plate and serving to drive a first adjusting screw that is in threaded fit with the adjusting press blocks, so that when the first adjusting screw rotates, the adjusting press blocks move along the adjusting slide, the adjusting press blocks having four lateral sides thereof formed with pressing ramps each pressing on one of the adjusting slide blocks, the adjusting slide blocks each being provided with a propping ramp that fits the corresponding pressing ramp and moves along the corresponding branch portion when being pressed, a deformable blocking portion being provided between the lateral sides of the adjacent two adjusting press blocks, the light shielding plate having an outer side provided with a receiving recess for receiving the deformable blocking portions that retract into the light shielding plate, each of the branch portions having a lateral surface provided with a radially extending returning groove, each of the adjusting slide blocks being provided with a returning bulge that moves along the returning groove, and a compression spring being arranged between the returning bulge and the returning groove for returning the adjusting slide block toward the adjusting press block.

3. The programmable graphic projection light source system of claim 1, further comprising a reflection angle adjusting device, which comprises a second micro servo motor, a screw seat, a screw nut, and adjusting arms, the second micro servo motor being fixed to the housing and abutting against a side of the reflective coating opposite the flexible screen, the second micro servo motor driving a second adjusting screw to move in a direction of light irradiating directly onto the pupil, the screw seat being fixed to the housing and in rotational fit with an end of the second adjusting screw opposite the second micro servo motor, the screw nut being mounted around the second adjusting screw and moving along an axis of the second adjusting screw when the second adjusting screw rotates, the screw nut being peripherally provided with at least four circumferentially distributed links, the adjusting arms being in one-to-one correspondence with the links, and a center of the adjusting arm being pivotally connected to an end portion of the corresponding link, the link having one end pivotally connected to the screw seat and an opposite end pivotally connected to the side of the reflective coating opposite the flexible screen.

4. The programmable graphic projection light source system of claim 1, wherein the flexible screen is connected to an image input device in a wired or wireless manner.
